(19) United States
(12) Patent Application Publication
(10) Pub. No.: US 2010/0185076 A1
(43) Pub. Date: Jul. 22, 2010

(54) GARMENT FOR MEASURING PHYSIOLOGICAL SIGNAL AND SYSTEM FOR PROCESSING PHYSIOLOGICAL SIGNAL

(75) Inventors: Yeon-Hee Jeong, Daejeon (KR); Young-Ju Jeon, Daejeon (KR); Seung-Chul Shin, Daejeon (KR); Yong-Won Jang, Daejeon (KR); Seung-Hwan Kim, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/669,023

(22) PCT Filed: May 13, 2008
(86) PCT No.: PCT/KR08/02658

§ 371 Date: Jan. 13, 2010

(30) Foreign Application Priority Data
Jul. 20, 2007 (KR) 10-2007-0073100

(51) Int. Cl. A61B 5/04

(52) U.S. Cl. 600/388

(57) ABSTRACT

Provided are a physiological signal measurement garment and a physiological signal processing system. The physiological signal measurement garment includes: a main garment body which is formed of an elastic fabric and includes a mesh structure and an elastic band; at least one physiological signal sensing electrode sewn on the garment body; a physiological signal transmission unit which is sewn on the garment body and transmits a physiological signal sensed by the physiological signal sensing electrode; and a physiological information measurement module that measures various kinds of physiological information from the physiological signal, which is sensed by the physiological signal sensing electrode and transmitted through the physiological signal transmission unit. The physiological signal measurement garment is more comfortable for a wearer than similar existing garments and can measure a physiological signal with higher accuracy.

### TECHNICAL FIELD

The present invention relates to physiological signal measurement and processing, and more particularly, to a physiological signal measurement garment which can improve the convenience for its wearer and can measure a physiological signal with higher accuracy, and a physiological signal processing system.

The present invention is derived from a research project by the Information Technology (IT) Research & Development (R&D) program of the Ministry of Information and Communication (MIC) and the Institute for Information Technology Advancement (IITA) [2006-S-007-01], Ubiquitous Health Monitoring Module and System Development].

### BACKGROUND ART

With developments in information and communication technology, the need for e-health care that enables the acquisition of physiological information in real time, anytime and anywhere, is increasing, and its application fields are diversifying. Thus, a large demand for bio-cloths, i.e., physiological signal measurement garments, which enable the acquisition and transmission of physiological information to a remote site, is expected over a wide range of fields, for example, for elderly care service, prompt on-site emergency treatment and prescription, body stability check-up for military personnel and workers in dangerous environments, and warning about dangerous situations.

Conventional research on physiological signal measurement garments is as follows. First, there is a method of using electrodes and/or sensors which can be selectively detached, wherein, for example, a velcro pad is used to detach an electrode/sensor. According to this method, electrodes and/or sensors can be attached to or detached from the garment, and the garment can stably contact the skin. In addition, the electrodes are coated with conductive silicon for conductivity. However, since electrodes have to be attached or detached in order to measure physiological signals, it would be inconvenient for a user who needs to carry out an urgent measurement.

Second, in a method of measuring physiological signals using an elastic band (U.S. Pat. No. 6,553,247), since the elastic band includes a fabric electrode, the elastic band may tighten around the body without skin troubles or discomfort. However, its band style design restricts how it can be worn in terms of style and application, and data which can be measured by using the elastic band is also limited.

Third, a method of using a fabric electrode which comprises multi-channel sensors and which is inserted into the garment through a transmission line in the form of a conductive string and then fixed to a module by a connector is disclosed (Korean Patent Application No. 2006-0018511). However, instability of the connector and instability in sensing due to bodily movement are pointed out as drawbacks of this method.

As described above, the conventional physiological signal measurement garments were designed without accurate understanding of bodily movements in daily life and have a number of limiting factors in terms of stably and accurately acquiring physiological information.

### DISCLOSURE OF INVENTION

### Technical Problem

The present invention provides a physiological signal measurement garment which can improve the convenience for a wearer and provides higher measurement accuracy.

The present invention provides a physiological signal processing system for processing a physiological signal measured by the physiological signal measurement garment.

### Technical Solution

According to an aspect of the present invention, there is provided a physiological signal measurement garment comprising: a garment body which is formed of an elastic fabric and includes a mesh structure and an elastic band; at least one physiological signal sensing electrode sewn on the garment body; a physiological signal transmission unit which is sewn on the garment body and transmits a physiological signal obtained by the physiological signal sensing electrode; and a physiological information measurement module which measures various kinds of physiological information from the physiological signal obtained by the physiological signal sensing electrode and transmitted through the physiological signal transmission unit.

According to another aspect of the present invention, there is provided a physiological signal measurement garment comprising: at least one physiological signal sensing electrode sewn on a garment body designed to fit to the body considering the shape of human muscles and their motional features; a transmission line that is sewn on the garment body and transmits a physiological signal sensed by the physiological signal sensing electrode; a transmission line cover that is sewn on the garment body and insulates the transmission line from the body; and a physiological signal transmission module which obtains the physiological signal, which is sensed by the physiological signal sensing electrode and transmitted through the transmission line, and transmits the collected physiological signal to an external device.

According to another aspect of the present invention, there is provided a physiological signal processing system comprising: a garment body that includes a mesh structure and an elastic band in an elastic fabric and includes at least one physiological signal sensing electrode and a physiological transmission unit transmitting a physiological signal sensed by the physiological signal sensing electrode, the physiological signal sensing electrode and the physiological signal transmission unit being sewn on the garment body; a physiological information measurement module that is attachable to and detachable from the garment body and measures various kinds of physiological information from the physiological signal, which is sensed by the physiological signal sensing electrode and transmitted through the physiological signal transmission unit; and a physiological information analysis module which analyzes the physiological information provided by the physiological information measurement module.

According to another aspect of the present invention, there is provided a physiological signal processing system comprising: a garment body that is designed to fit to the body considering the shape of human muscles and their motional features and on which at least one physiological signal sensing electrode, a transmission line which transmits a physiological signal sensed by the physiological signal sensing electrode, and a transmission line cover which insulates the transmission line from the body are sewn; a physiological signal transmission module which transmits the physiological signal sensed by the physiological signal sensing electrode and transmitted through the transmission line; and a physiological information measurement/analysis module which measures and analyzes various kinds of physiological information from the physiological signal transmitted from the physiological signal transmission module.

### DESCRIPTION OF DRAWINGS

The above and other features and advantages of the present invention will become more apparent by describing in detail exemplary embodiments thereof with reference to the attached drawings in which:

FIG. 1 illustrates a structure of a physiological signal measurement garment according to an embodiment of the present invention;

FIGS. 2A and 2B are front and rear views of the physiological signal measurement garment of FIG. 1, respectively;

FIGS. 3A and 3B illustrate structures of a transmission line, a transmission line cover, a physiological signal measurement electrode sewn on the transmission line, and a connector in the physiological signal measurement garment of FIG. 1;

FIG. 4 is a sectional view of part of the physiological signal measurement garment of FIG. 1 for explaining how components thereof are connected;

FIGS. 5A and 5B are views for explaining the connection of the connector in the physiological signal measurement garment of FIG. 1;

FIG. 6 illustrates an example of the physiological signal measurement garment in FIG. 1; and

FIGS. 7A and 7B are block diagrams of physiological signal processing systems according to embodiments of the present invention.

### MODE FOR INVENTION

Hereinafter, exemplary embodiments of a physiological signal measurement garment and a physiological signal processing signal according to the present invention will be described in detail with reference to the appended drawings.

Prior to giving a description of a physiological signal measurement garment and a physiological signal processing system according to the present invention, human muscle structure and features of muscular motion will be briefly described.

The human body uses a number of muscles as it moves. The muscles move in a pre-determined direction along with the skin surface as the body moves. A garment, which is put directly on the surface of a person's skin, can be made to be as stable as possible if the shape of muscular groups and their motion according to bodily movement are considered. Especially, when fabricating a physiological signal measurement garment which uses a fabric electrode, there is a need to minimize the friction of the fabric electrode against the skin surface when the body moves. To this end, the garment is designed to have a buffer structure which prevents the motion of muscles and skin from affecting other portions in the garment. In other words, elastic seams are formed in the garment by considering the muscular shapes, and an elastic mesh structure is inserted for a buffering action so that the muscular and skin motion can be sufficiently absorbed. In particular, on the front of the garment, elastic seams are formed to correspond to the boundaries of the trapezius muscle, pectoralis major muscle, rectus abdominis muscle, and external oblique abdominal muscle, and an elastic mesh is inserted. In addition, considering the motion of upper arms, an elastic band is inserted into a part over the serratus anterior muscle and the external oblique abdominal muscle. In addition, in order to minimize the motion of the pectoralis major muscle as the upper arms move, a buffering elastic mesh structure is inserted into an elastic seam near the armpit. On the rear of the garment, according to the same principle as applied to the front of the garment, elastic seams are formed to correspond to the boundaries of the teres major muscle, trapezius muscle, and teres minor muscle, and an elastic mesh structure is inserted. This design of the garment ensures a tighter contact between the skin and electrodes, maintains stable contact between the skin and the electrodes even when the body moves, and reduces the contact resistance between the skin and the electrodes, thereby reducing noise, which is generated when sensing/monitoring physiological signals.

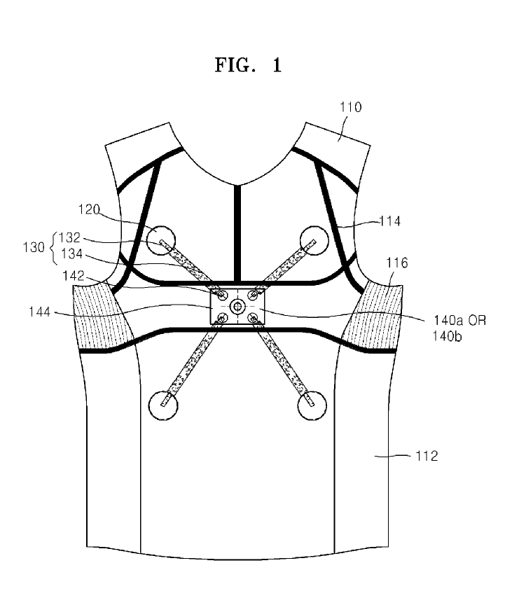

FIG. 1 illustrates a structure of a physiological signal measurement garment according to an embodiment of the present invention. The physiological signal measurement garment in FIG. 1 includes a garment body 110 that is formed of an elastic fabric and includes a mesh structure and an elastic band, at least one physiological signal sensing electrode 120 sewn on the garment body 110, a physiological signal transmission unit 130, which is sewn on the garment body 110 and transmits a physiological signal obtained from the physiological signal sensing electrode 120, and a physiological signal measurement module 140a measuring various kinds of physiological information from the physiological signal obtained from the physiological signal sensing electrode 120 and transmitted through the physiological signal transmission unit 130. Alternatively, instead of the physiological signal transmission unit 130 and the physiological signal measurement module 140a, a physiological signal transmission module 140b, which obtains the physiological signal from the physiological signal sensing electrode 120 and externally transmits it by wire or wirelessly, may be included. In this case, an external device connected to the garment by wire or wirelessly may more accurately measure physiological information.

Referring to FIG. 1, the garment body 110 is fabricated to reflect the shape of human muscles and features of muscular motion. In particular, the garment body 110 is formed of an elastic, tightening material 112, and includes a mesh structure 114 formed along elastic seams and an elastic band 116 locally inserted in the garment body 110. The tightening material 112 is used to ensure tight contact with the body and comfortable bodily movement, and the mesh structure 114 is used to reflect the shape of human muscles and features of muscular motion. The elastic band 116 protects the physiological signal sensing electrode 120 and the physiological information measurement module 140a or the physiological signal transmission module 140b from strenuous body motions.

The physiological signal sensing electrode 120 is formed of a conductive fabric, which is sewn on the garment body 110. For example, the conductive fabric may be silver-plated or silver-coated fabric that does not adversely affect human skin. The physiological signal transmission unit 130 includes a transmission line 132 and a transmission line cover 134. The transmission line 132 is formed of a conductive elastic band and connects the physiological signal sensing electrode 120 and the physiological information measurement module 140a or the physiological signal transmission module 140b. The transmission line cover 134 insulates the transmission line 132 from the skin and simultaneously fixes the transmission line 134 to the garment body 110.

The physiological information measurement module 140a or the physiological signal transmission module 140b includes a connector 142 for connection to the transmission line 132 and a module case 144.

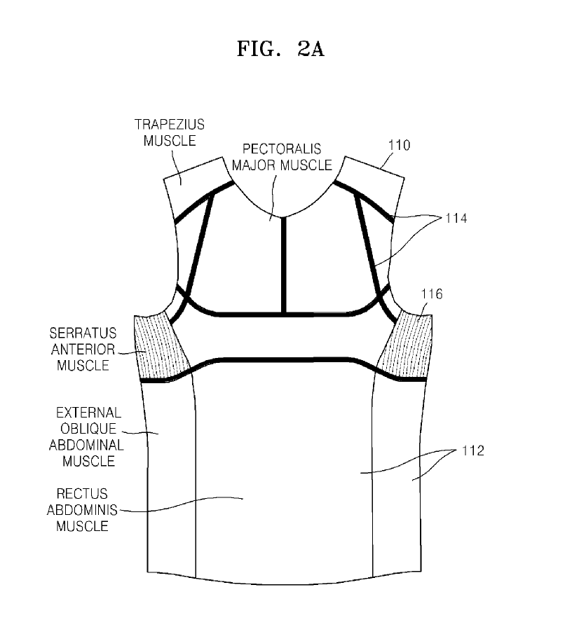

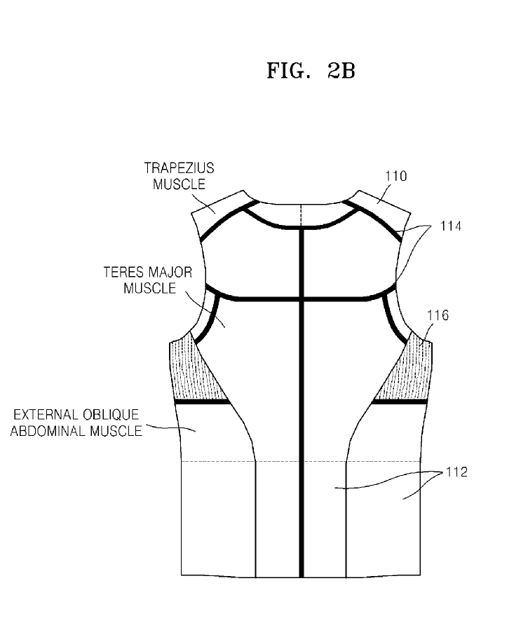

FIGS. 2A and 2B are front and rear views of the physiological signal measurement garment of FIG. 1, respectively. The garment body 110 is formed of an elastic tightening material 112, such as tricot. In the garment body 110, the mesh structure 114 is arranged along each elastic seam considering the shape of human muscles and features of muscular motion, and the elastic band 116 is arranged under the arms in order to minimize movement of the electrodes and the module, which may occur as the body moves.

Referring to FIG. 2A, the shape of the human muscles is divided into trapezius muscle, pectoralis major muscle, rectus abdominis muscle, and external oblique abdominal muscle, and the elastic seams are formed to correspond to the boundaries of these muscles. The elastic mesh structure 114 is arranged along each of the elastic seams for buffering functions. Here, the mesh structure 114 for each elastic seam may be formed of the same material or a different material considering the motion of a specific muscle. In addition, considering the motion of the upper arms, the elastic band 116 is arranged near the serratus anterior muscle, i.e., under the arms. In particular, the parts corresponding to the external oblique abdominal muscle and the rectus abdominis muscle are formed of an elastic material in order to enhance the tight contact with the body. In addition, in order to minimize the motion of the electrodes when the pectoralis major muscle flex as the upper arms move, the elastic mesh structure 114 is arranged along the elastic seams near the arm holes of the garment.

Referring to FIG. 2B, according to the same principle applied to the front upper body in FIG. 2A, the shape of human muscles is divided into the teres major muscle, trapezius muscle, and teres minor muscle, and the elastic seams are formed to correspond to the boundaries of these muscles. The elastic mesh structure 114 is arranged along each of the elastic seams and have a buffering function.

As described above, the design of the garment ensures tighter contact between the skin and electrodes, maintains stable contact between the skin and the electrodes even when the body moves, and reduces contact resistance between the skin and the electrodes, thereby reducing noise, which is generated when sensing/monitoring physiological signals.

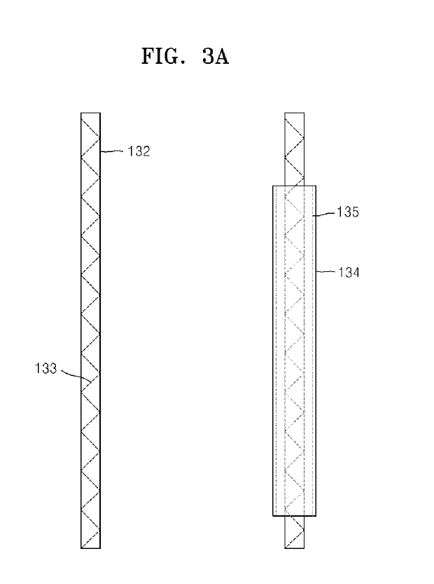

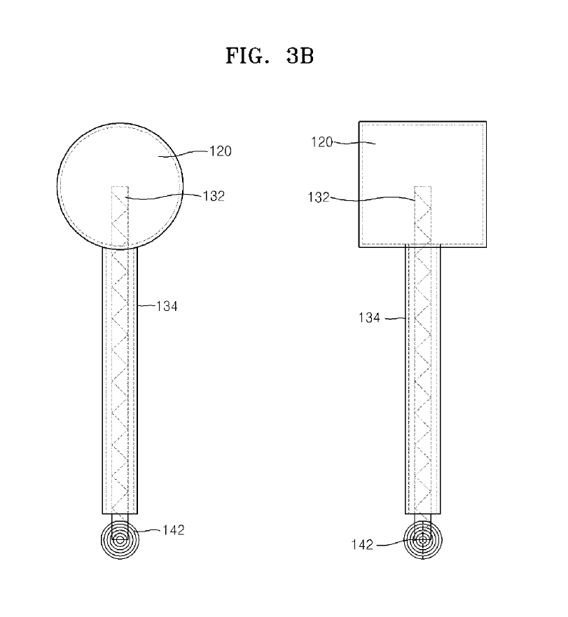

FIGS. 3A and 3B illustrate structures of the transmission line 132, the transmission line cover 134, the physiological signal measurement electrode 120 sewn on the transmission line 132, and the connector 142 in the physiological signal measurement garment of FIG. 1.

Referring to FIG. 3A, the transmission line 132 is a conductive elastic band and is sewn on the garment body 110 in a zigzag fashion considering the tight contact with the garment body 110, elasticity and stability. In addition, the transmission line cover 134 is formed of an elastic fabric and prevents direct contact between the transmission line 132 and the skin. The transmission line cover 134 is sewn on the garment body 110 to have a tunnel structure 135. Due to this structure, a physiological signal can be stably transmitted even when the body moves, and the transmission line 132 can retain the same elasticity as the material of the garment body 110 itself, thereby providing comfort to a wearer and enabling the stable acquisition of a physiological signal.

Referring to FIG. 3B, the physiological signal sensing electrode 120, i.e., a fabric electrode, is sewn on the transmission line 132. In an embodiment, the physiological signal sensing electrode 120 may have a diameter of 25-35 mm. Here, the physiological signal sensing electrode 120 may have various shapes, for example, a rectangular or circular shape. The physiological signal obtained by the physiological signal sensing electrode 120 is transmitted to the transmission line 132, which is insulated by the transmission line cover 134, is obtained by the connector 142 having a diameter of, for example, 7 mm, and is then transmitted to the physiological information measurement module 140a or the physiological signal transmission module 140b. The connector 142 is a snap button type connector and is detachable. In addition, its fixedness and stability are robust enough to stably transmit physiological information despite a wearer's strenuous motions, such as running. The size of this snap button type connector 142 can be varied according to the use of the garment and can be easily manufactured to order and is widely available.

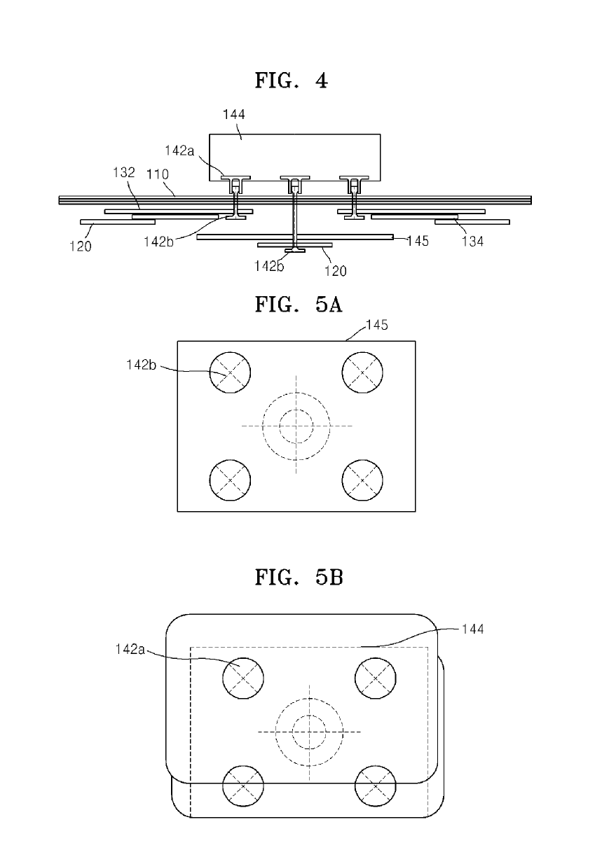

FIG. 4 is a sectional view of part of the physiological signal measurement garment of FIG. 1 for explaining how components thereof are connected.

Referring to FIG. 4, the garment body 110, which is designed considering the shape of human muscles and their motional features, is combined with the physiological signal sensing electrode 120, the transmission line 132 transmitting the physiological signal obtained by the physiological signal sensing electrode 120, the transmission line cover 134, which insulates the transmission line 132 from the skin, a connector cover 145, which insulates the connector 142 from the skin, the connector 142, which includes a recess connector member 142a connected to the physiological information measurement module 140a or the physiological signal transmission module 140b, and a protrusion connector member 142b, and the module case 144 accommodating the physiological information measurement module 140a or the physiological signal transmission module 140b. The connector 142 can be attached or detached according to the body part to be measured or the number of channels. In other words, the connector 142 can be easily and conveniently attached to various kinds of garments together with a detachable module.

FIGS. 5A and 5B are views for explaining the connection of the connector 142 in the physiological signal measurement garment of FIG. 1. FIG. 5A illustrates the arrangement of the protrusion connector members 142b, which are attached to the garment body 110, on the connector cover 145. The arrangement of the connector 142 may be varied. FIG. 5B illustrates the recess connector members 142a attached to the module case 144. The recess connector members 142a are coupled to the protrusion connector members 142b, which are attached to the garment body 110, respectively, and receive the physiological signal transmitted from the physiological signal sensing electrode 120.

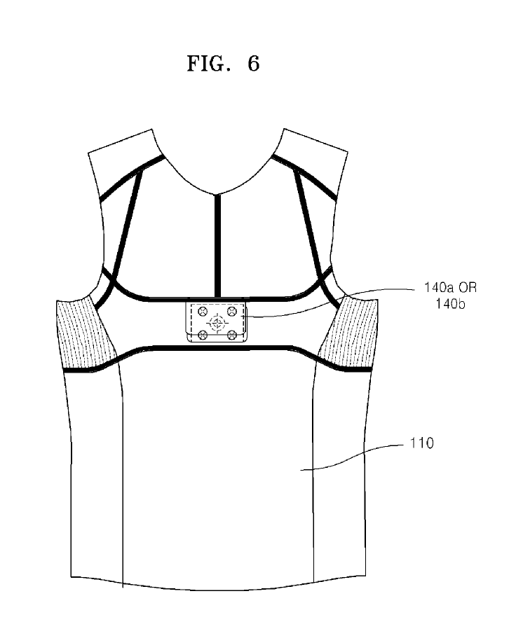

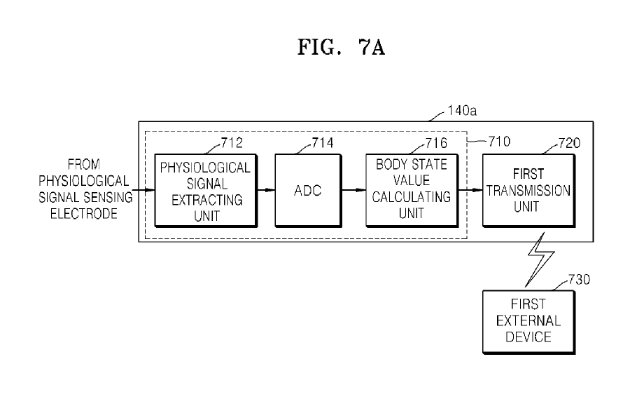

FIG. 6 illustrates an example of the physiological signal measurement garment of FIG. 1. Referring to FIG. 6, the physiological information measurement module 140a or the physiological signal transmission module 140b is attached to the garment body 110, which is ergonomically designed. The location of the physiological information measurement module 140a or the physiological signal transmission module 140b may be varied.

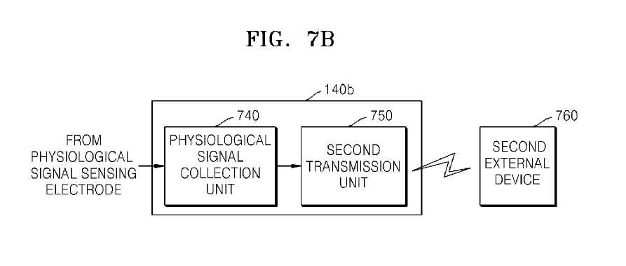

FIGS. 7A and 7B are block diagrams of physiological signal processing systems according to embodiments of the present invention.

A physiological signal processing system illustrated in FIG. 7A includes a physiological information measurement module 140a and a first external device 730. The physiological information measurement module 140a is constructed to be attachable to or detachable from the garment body 110. The physiological information measurement module 140a can be attached to or detached from the garment body 110 by using the snap button type connector 142. The physiological information measurement module 140a includes a physiological information acquisition unit 710, which acquires physiological information from the physiological signal transmitted from the physiological signal sensing electrode 120, and a first transmission unit 720. The physiological signal acquisition unit 710 includes a physiological signal extracting unit 712, an analog-to-digital converter (ADC) 714, and a body state value calculating unit 716.

The physiological signal extracting unit 712 extracts analog data, such as an electrocardiogram (ECG), a breathing waveform, and body temperature, from the physiological signal transmitted from the physiological signal sensing electrode 120 through the transmission line 132. Here, the physiological signal sensed by the physiological signal sensing electrode 120 has a very low magnitude, and thus an amplification process can be performed to ensure higher accuracy in physiological signal measurement. In addition, in order to remove noise included in the amplified physiological signal, a filtering process may be performed. In other words, the physiological signal extracting unit 712 may include an amplifying circuit and/or a filter circuit. In this case, the physiological signal extracting unit 712 extracts the analog data after amplifying the transmitted physiological signal to a measurable amplitude and/or filtering the amplified physiological signal.

The ADC 714 converts the analog data extracted by the physiological signal extracting unit 712 to digital data. The body state value calculating unit 716 calculates body state values, i.e., physiological information, such as a heart rate, a breathing rate, the amount of exercise performed, exercise distance, exercise speed, calories burned, and body temperature, from the digital data provided from the ADC 714.

The first transmission unit 720 transmits the physiological information acquired by the physiological information acquisition unit 710 to the first external device 730 by wired communication or wireless communication using, for example, wireless Internet or Bluetooth.

The first external device 730 is a physiological information analysis module and may include a wearer's mobile device or a computer installed at a remote site, such as a hospital or a health center. The first external device 730 analyzes the physiological information provided from the physiological information measurement module 140a and determines the health status of the wearer. The first external device 730 displays the result of the determination on the mobile device or outputs the result as an audio signal. In addition, the result of the determination may be fed back to a registered computer of the wearer. Alternatively, the physiological information analysis module, i.e., the first external device 730, may be installed in the physiological information measurement module 140a.

A physiological signal processing system in FIG. 7B includes a physiological signal transmission module 140b, which is attached to the garment body 110, and a second external device 760. The physiological signal transmission module 140b is constructed so as to be attachable to or detachable from the garment body 110. The physiological signal transmission module 140b can be attached to or detached from the garment body 110 by using the snap button type connector 142. The physiological signal transmission module 140b includes a physiological signal collection unit 740 and a second transmission unit 750.

The physiological signal collection unit 740 collects the physiological signal transmitted from the physiological signal sensing electrode 120. The second transmission unit 750 transmits the physiological signal collected by the physiological signal collection unit 740 to the second external device 760 by wired communication or wireless communication.

The second external device 760 is a physiological information measurement/analysis module and acquires physiological information by processing the transmitted physiological signal. In other words, the second external device 760 performs the operation of the first external device 730 in FIG. 7A as well as the operations of the physiological signal extracting unit 712, the ADC 714 and the body state value calculating unit 716.

Although processing of an ECG, a breathing rate, acceleration of heart-beat, and temperature data is described as an example, other physiological signal measuring circuits, such as for measuring blood pressure and oxygen saturation, can be added.

According to the present invention, a physiological signal measurement garment is designed considering the shape of human muscles and their motional features, so that accurate physiological information can be acquired and transmitted while not causing discomfort to the wearer involved in various activities. In addition, the physiological signal measurement garment is formed using various ergonomic materials to fit to the body, thereby enhancing the comfort of the wearer. Furthermore, the physiological signal measurement garment applies appropriate pressures on particular body sites, thereby ensuring stable contact between the physiological signal sensing electrode and the body, with the expectation of improving exercise capacity, for example, muscle fatigue reduction during exercise. The design of the physiological signal measurement garment based on the shape of human muscles also provides an aesthetic effect.

In addition, according to the present invention, by using the physiological signal sensing electrode formed of a fabric electrode, the transmission line formed of a conductive elastic band, and the snap button type connector, the physiological signal can be stably transmitted to a module and can be directly transmitted to an external device. In other words, the module can be detached from the physiological signal measurement garment without an additional device. In particular, the fabric electrode and the translation line with the insulating structure ensure the acquisition of stable physiological signals and transmission of stable physiological information no matter what kind of motion or activity the wearer is involved in. The shape, size and number of fabric electrodes, the shape and size of the conductive elastic transmission line, and the shape, size and location of the connector may be widely varied according to the use and function of the garment.

In addition, the fabric electrode, the transmission line, the connector and the module which are attached to the physiological signal measurement garment are constructed so that the sensed physiological signal and information on the physiological signal can be wirelessly transmitted and the contact between the electrode and skin can be stably maintained in daily life. As a result, the physiological signal can be monitored for a long time. This physiological signal measurement garment according to the present invention can be used to check the current health status and for continuous, regular medical checkups, which is useful for systematic health management and preventing disease. Furthermore, for athletes and those who enjoy active leisure activities, the physiological signal measurement garment according to the present invention can be used to analyze physiological signal variations according to the exercise load, thereby improving their exercise capacity. In addition, for those who work in dangerous environments, such as firemen, policemen, and military personnel, the physiological signal measurement garment according to the present invention can be used to assess an emergency situation and save lives.

The above-described present invention may be implemented with at least one processor and includes a computer readable medium including a program instruction for executing various operations realized by a computer. The computer readable medium may include a program instruction, a data file, and a data structure, separately or cooperatively. The program instructions and the media may be those specially designed and constructed for the purposes of the present invention, or they may be of the kind well known and available to those of ordinary skill in the art of computer software arts. Examples of the computer readable media include magnetic media (e.g., hard disks, floppy disks, and magnetic tapes), optical media (e.g., CD-ROMs or DVD), magneto-optical media (e.g., floptical disks), and hardware devices (e.g., ROMs, RAMs, or flash memories, etc.) that are specially configured to store and perform program instructions. The media may also be transmission media such as optical or metallic lines, wave guides, etc. including a carrier wave transmitting signals specifying the program instructions, data structures, etc. Examples of the program instructions include both machine code, such as produced by a compiler, and files containing high-level languages codes that may be executed by the computer using an interpreter.

While this invention has been particularly shown and described with reference to exemplary embodiments thereof, it will be understood by those of ordinary skill in the art that various changes in form and details may be made therein without departing from the spirit and scope of the invention as defined by the appended claims. The exemplary embodiments should be considered in a descriptive sense only and not for purposes of limitation. Therefore, the scope of the invention is defined not by the detailed description of the invention but by the appended claims, and all differences within the scope will be construed as being included in the present invention.

1. A physiological signal measurement garment comprising:
a garment body which is formed of an elastic fabric and includes a mesh structure and an elastic band;
at least one physiological signal sensing electrode sewn on the garment body;
a physiological signal transmission unit which is sewn on the garment body and transmits a physiological signal obtained by the physiological signal sensing electrode; and
a physiological information measurement module which measures various kinds of physiological information from the physiological signal obtained by the physiological signal sensing electrode and transmitted through the physiological signal transmission unit.

2. The physiological signal measurement garment of claim 1, wherein the physiological signal sensing electrode is formed of one of a conductive fabric and a fabric coated with a metal which is safe for human skin to contact.

3. The physiological signal measurement garment of claim 1, wherein the physiological signal transmission unit comprises:
a transmission line which transmits the physiological signal sensed by the physiological signal sensing electrode to the physiological information measurement module; and
a transmission line cover which insulates the transmission line from the skin.

4. The physiological signal measurement garment of claim 3, wherein the transmission line is formed of a conductive elastic band and is sewn on the garment body in a zigzag fashion.

5. (canceled)

6. The physiological signal measurement garment of claim 1, wherein the physiological information measurement module is attachable to and detachable from the garment body and is connected to the physiological signal transmission unit by using a snap button type connector.

7. The physiological signal measurement garment of claim 1, wherein the physiological information measurement module comprises:
a physiological information acquisition unit which acquires physiological information from the physiological signal sensed by the physiological signal sensing electrode and transmitted through the physiological signal transmission unit; and
a transmission unit which transmits the physiological information acquired by the physiological information acquisition unit to an external device.

8. The physiological signal measurement garment of claim 1, wherein the mesh structure in the garment body is designed according to the shape of human muscles.

9. A physiological signal measurement garment comprising:
at least one physiological signal sensing electrode sewn on a garment body designed to fit to the body considering the shape of human muscles and their motional features;
a transmission line that is sewn on the garment body and transmits a physiological signal sensed by the physiological signal sensing electrode;
a transmission line cover that is sewn on the garment body and insulates the transmission line from the body; and
a physiological signal transmission module which obtains the physiological signal, which is sensed by the physiological signal sensing electrode and transmitted through the transmission line, and transmits the collected physiological signal to an external device.

10. The physiological signal measurement garment of claim 9, wherein the physiological signal sensing electrode is formed of one of a conductive fabric and a fabric coated with a metal which is safe for human skin to contact.

11. The physiological signal measurement garment of claim 9, wherein the transmission line is formed of a conductive elastic band and is sewn on the garment body in a zigzag fashion.

12. The physiological signal measurement garment of claim 9, wherein the transmission line cover is formed of an insulating elastic band and is sewn on the garment body to have a tunnel structure.

13. The physiological signal measurement garment of claim 9, wherein the physiological signal transmission module is attachable to and detachable from the garment body and is connected to the transmission line by using a snap button type connector.

14. The physiological signal measurement garment of claim 9, wherein the physiological signal transmission module comprises:
a physiological signal collection unit which collects the physiological signal sensed by the physiological signal sensing electrode and transmitted through the transmission line; and
a transmission unit which transmits the physiological signal obtained by the physiological signal obtaining unit to an external device.

15. A physiological signal processing system comprising:
a garment body that includes a mesh structure and an elastic band in an elastic fabric and includes at least one physiological signal sensing electrode and a physiological transmission unit transmitting a physiological signal sensed by the physiological signal sensing electrode, the physiological signal sensing electrode and the physiological signal transmission unit being sewn on the garment body;
a physiological information measurement module that is attachable to and detachable from the garment body and measures various kinds of physiological information from the physiological signal, which is sensed by the physiological signal sensing electrode and transmitted through the physiological signal transmission unit; and
a physiological information analysis module which analyzes the physiological information provided by the physiological information measurement module.

16. The physiological signal processing system of claim 15, wherein the physiological signal transmission unit comprises a transmission line formed of a conductive elastic band, and a transmission line cover.

17. The physiological signal processing system of claim 15, wherein the physiological information measurement module comprises:
a physiological information acquisition unit which acquires physiological information from the physiological signal sensed by the physiological signal sensing electrode and transmitted through the physiological signal transmission unit; and
a transmission unit which transmits the physiological information acquired by the physiological information acquisition unit to an external device.

18. The physiological signal measurement garment of claim 15, wherein the mesh structure in the garment body is designed according to the shape of human muscles.

19. A physiological signal processing system comprising:
a garment body that is designed to fit to the body considering the shape of human muscles and their motional features and on which at least one physiological signal sensing electrode, a transmission line transmitting a physiological signal sensed by the physiological signal sensing electrode, and a transmission line cover insulating the transmission line from the body are sewn;
a physiological signal transmission module which transmits the physiological signal sensed by the physiological signal sensing electrode and transmitted through the transmission line; and
a physiological information measurement/analysis module which measures and analyzes various kinds of physiological information from the physiological signal transmitted from the physiological signal transmission module.

20. The physiological signal processing system of claim 19, wherein the physiological signal sensing electrode is formed of one of a conductive fabric and a fabric coated with a metal which is safe for human skin to contact.

21. The physiological signal processing system of claim 19, wherein the physiological signal transmission module comprises:
a physiological signal collection unit which collects the physiological signal sensed by the physiological signal sensing electrode and transmitted through the transmission line; and
a transmission unit which transmits the physiological signal collected by the physiological signal obtaining unit to the physiological information measurement/analysis module.
